(19) United States
(12) Patent Application Publication
(10) Pub. No.: US 2019/0307576 A1
(43) Pub. Date: Oct. 10, 2019

(54) EXPANDABLE CORPECTOMY IMPLANT

(71) Applicants: Matthew F. Philips, M.D., Dartmouth, MA (US); Miles Wilson, Tiverton, RI (US); Brandon B. Arthurs, Wilmington, NC (US)

(72) Inventors: Matthew F. Philips, M.D., Dartmouth, MA (US); Miles Wilson, Tiverton, RI (US); Brandon B. Arthurs, Wilmington, NC (US)

(21) Appl. No.: 16/375,481

(22) Filed: Apr. 4, 2019

Related U.S. Application Data
(60) Provisional application No. 62/652,771, filed on Apr. 4, 2018.

(51) Int. Cl. A61F 2/44

(52) U.S. Cl. CPC A61F 2/4455; A61F 2002/30601

(57) ABSTRACT

An expandable corpectomy implant and methods of making and using same. The expandable spinal implant includes an inner member and an outer member. The inner member and outer member each have a first end, a second end and a sidewall. The inner member is positioned in the outer member and the inner member and the outer member have a selected cross sectional shape. The inner member is configured such that it slidably protrudes from the outer member. The inner member has at least one tab incorporated into the sidewall of the inner member and the outer member has at least one aperture formed in the sidewall of the outer member such that the at least one tab of the inner member engages the corresponding at least one aperture of the outer member so that the inner member is locked with the outer member.

### CROSS-REFERENCE TO RELATED APPLICATIONS

The present application claims the benefit under 35 U.S.C. § 119(e) of U.S. Provisional application Ser. No. 62/652,771, filed Apr. 4, 2018, entitled “Expandable Corpectomy Implant”, which is hereby expressly incorporated herein in its entirety.

### BACKGROUND

Spinal implants often assume a particular shape due to reasons associated with manufacturing or limitations of placement during the surgical approach. For instance, implants utilized in association with corpectomy, a surgical procedure to remove a vertebral body, are often manufactured by manipulating a single mass. This type of manufacturing leads to limitations associated with the cross-sectional shape such an implant can assume.

A drawback to cross-sectional shape limitations associated with corpectomy implants relates to subsidence. Subsidence, the sinking or settling of an implant into bone, lead to sub-optimal outcomes following surgery. Complications related to subsidence often include loosening of screws, loosening of implants, and suboptimal bone healing. Several studies have demonstrated that wider implants with larger footprints provide more resistance to subsidence, though the methods associated with the design and manufacture of prior art corpectomy implants have limited the ability of corpectomy cages to assume selected, wider shapes. This predicament relates particularly to corpectomy cages having an expandable nature.

The strongest bone section of a vertebral body is the epiphyseal ring. Where possible, it is preferable for any surgical implant utilized in association with spinal fusion surgery or corpectomy surgery to maximize contact with the endplate at the epiphyseal ring instead of relying on contact with the portion of the endplate contained within the epiphyseal ring, which contains softer bone, and is a more likely point of subsidence. Previously known expandable corpectomy cages, however, are generally only circular in cross-sectional shape and thereby lack the variety of cross-sectional shapes and sizes to provide for adequate or preferable contact with the epiphyseal ring following implantation, and are therefore disadvantageously prone to subsidence-related complications.

Prior art cages used in association with corpectomy procedures utilize internal distraction mechanisms. Such distraction mechanisms require the filling of the volume within the implant to accommodate distraction mechanisms, thereby taking precious space that otherwise could be filled with biological material that might improve the likelihood of bone healing and thereby improve patient outcomes.

Moreover, prior art distraction mechanisms utilized in association with corpectomy require the rotation of one or more of the components of prior art implants utilized in association with corpectomy procedures. The requirement of rotation often limits the variety of cross-sectional shapes and sizes that may be utilized in association with implants for corpectomy procedures. For instance, many prior art expandable implants that require rotation in association with expansion must assume a substantially round or circular cross-sectional form, thereby limiting the ability for the prior art expandable implants to maintain adequate contact with the epiphyseal ring or to maintain adequate endplate area contact during intended use. Prior art expandable corpectomy implants have a rotating, threaded expansion method. Such a mechanism requires the cross-sectional shape of the implant cross-sectional implant to be round. Moreover, in such prior art implants, the square threads used can be relatively thick, which can increase wall thickness and decrease the internal volume available for bone graft placement. Further, a round or circular cross-sectional shape is often not ideal to fit the contours or shapes of a vertebral body and/or endplate.

Prior art spinal implants often have disadvantages associated with the method of manufacture. For instance, many prior art expandable corpectomy cages are machined from solid material structures. As a result, limited area exists in the outer wall to facilitate bone growth within and through the implant.

Prior art expandable corpectomy implants often use set screws to lock distraction in place and prevent loss of distraction. Such features can be relatively thick, which can increase wall thickness and decrease graft opening. Moreover, such mechanisms can be difficult to work with. Given the tight spaces that spine surgeons must routinely navigate, difficulty in utilization can increase the time required for and risks associated with corpectomy surgery.

### SUMMARY

Various embodiments of the present disclosure are generally directed to surgical implants, such as, but not limited to implants utilized in association with spine surgery.

In accordance with some embodiments, the spinal implant is of an expandable nature and related to a corpectomy procedure.

In accordance with some embodiments, an expandable corpectomy implant includes an inner member and an outer member. The inner member is of a shape and size to fit within the outer member. In some embodiments, the inner member incorporates one or more tabs. In one embodiment, the tab is plastic and deformable. Each of the plastically deformable tabs incorporates a tab platform and a taper. In some embodiments, the outer member includes a tab aperture of a corresponding shape to accommodate a tab platform. In some embodiments, both the inner member and outer member have a composition wherein the primary material is medical-grade titanium.

These and other features which may characterize various embodiments can be understood in view of the following detailed discussion and the accompanying drawings.

### BRIEF DESCRIPTION OF THE DRAWINGS

FIG. 1A is a sagittal view of an expandable corpectomy implant constructed in accordance with various embodiments.

FIG. 1B is an axial view of FIG. 1A.

FIG. 2A is a sagittal view of another embodiment of an expandable corpectomy implant.

FIG. 2B is an axial view of FIG. 2A.

FIG. 3A is a sagittal view of another embodiment of an expandable corpectomy implant.

FIG. 3B is an axial view of FIG. 3A.

FIG. 4A is a sagittal view of another embodiment of an expandable corpectomy implant.

FIG. 4B is an axial view of FIG. 4A.

### DETAILED DESCRIPTION

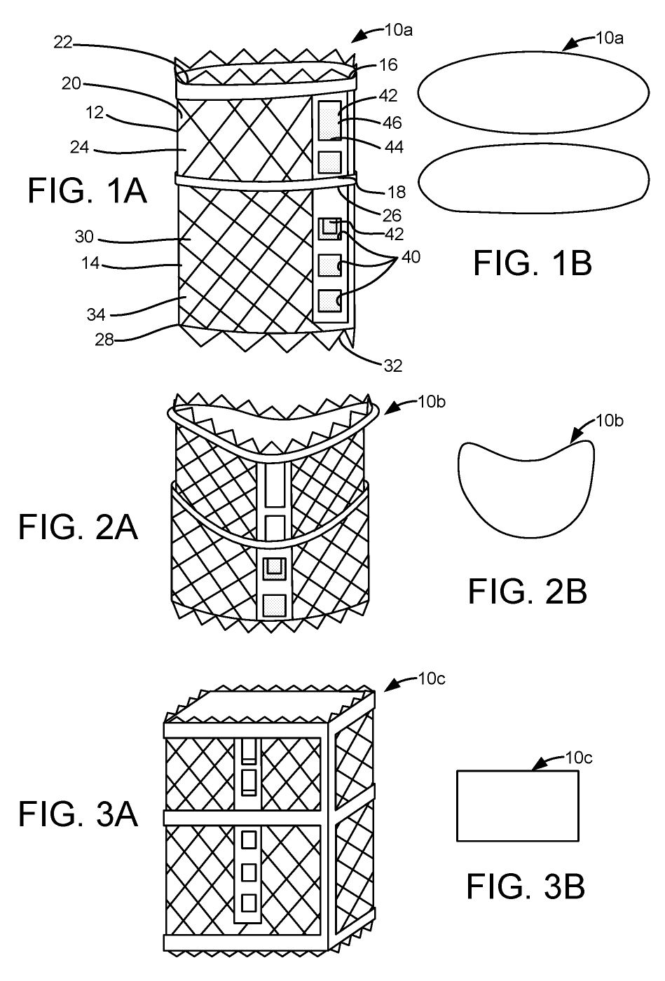

Referring now to the drawings, and more particularly to FIGS. 1A-4B, an expandable corpectomy implant 10 (10a, 10b, 10c, 10d) constructed in accordance with the present disclosure is shown. The implant 10 includes an inner member 12 and an outer member 14. The inner member 12 of the expandable corpectomy implant 10 has a first end 16, a second end 18 and a sidewall 20 having an inner surface 22 and an outer surface 24. The outer member 14 of the expandable corpectomy implant 10 has a first end 26, a second end 28 and a sidewall 30 having an inner surface 32 and an outer surface 34. In embodiments, the inner member 12 is configured such that it slidably protrudes outward from the outer member 14. In an embodiment, the axially protruding action takes place such that the overall height of the expandable corpectomy implant 10 increases as the inner member 12 protrudes axially from the outer member 14, as depicted in FIGS. 1.1-1.4. In some embodiments, the axially protruding action may be referred to as the expansion of the height of expandable corpectomy implant 10. In various embodiments, the height of the compressed, non-expanded expandable corpectomy implant 10 is approximately 8 millimeters in thoracic and lumbar applications of the expandable corpectomy implant, and approximately 10 millimeters in cervical applications of the expandable corpectomy implant 10.

In various embodiments, the axially protruding action of the inner member 12 sliding outward from the outer member 14 may expand the height of the expandable corpectomy implant 10 in discrete, locked increments corresponding to the locations of tab apertures 40 contained within the outer member 14, up to a maximum height of approximately 30 millimeters in thoracic and lumbar applications and to a maximum height of approximately 65 millimeters in cervical applications.

In various embodiments of the invention, the diameter of the expandable corpectomy implant's 10 cross-sectional shape orthogonal to the height axis ranges from approximately 10 millimeters to 35 millimeters. In embodiments of the expandable corpectomy implant 10 directed to lumbar and thoracic applications, variants may have a diameter ranging from approximately 19-30 millimeters. In embodiments of the expandable corpectomy implant 10 directed to cervical applications, variants may have a diameter ranging from approximately 12-16 millimeters. In embodiments, such diameters refer to the portion of the expandable corpectomy implant 10 placed substantially parallel to the plane of the vertebral body endplate, as one skilled in the art recognizes.

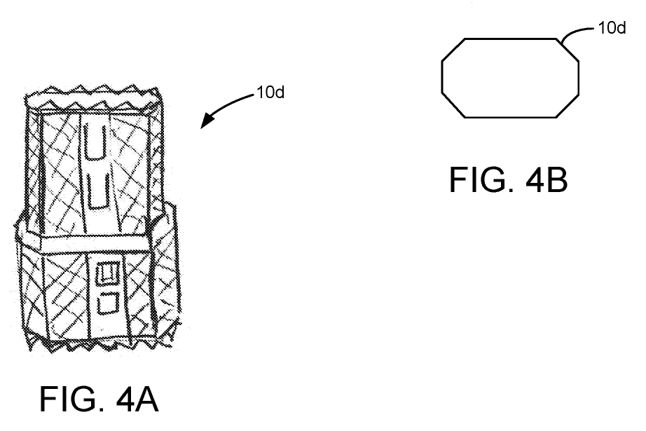

In various embodiments, the cross-sectional shape of the expandable corpectomy implant 10 can assume a variety of shapes and sizes. In embodiments, both the inner member 12 and the outer member 14 are configured such that they do not require rotation relative to the other during intended usage. One advantage of the non-rotating configuration is that, unlike prior art implants used in association with corpectomy, the lack of rotation allows for any geometrical or anatomically desirable footprint. In various embodiments, the cross-sectional shape of expandable corpectomy implant 10 assumes a shape and size specifically intended to maximize contact with one or more epiphyseal rings during intended usage. In an embodiment of the invention, the cross-sectional shape of the expandable corpectomy implant orthogonal to the height axis assumes the form of a square. In another embodiment of the invention, the cross-sectional shape of the expandable corpectomy implant orthogonal to the height axis assumes the form of a hexagon. In another embodiment of the invention, the cross-sectional shape of the expandable corpectomy implant orthogonal to the height axis assumes the form of an oval (FIGS. 1A-1B), a vertebra (FIG. 2A-2B), a rectangle (FIGS. 3A-3B) or an oxagon (FIGS. 4A-4B). In another embodiment of the invention, the cross-sectional shape of the expandable corpectomy implant orthogonal to the height axis assumes the form of a kidney. It will be understood by one of ordinary skill in the art that the expandable corpectomy implant can assume any shape. In various embodiments, the cross-sectional shape is evident in both the inner member 12 and outer member 14 of the expandable corpectomy implant 10 and configured such that the cross-sectional shape of the inner member is slightly smaller than but still corresponding to the cross-sectional shape of the outer member such that the inner member may slide within the outer member along the height axis.

In various embodiments, one or more plastically deformable tabs 42 are incorporated in the wall 20 of the inner member 12, as depicted in FIGS. 1A-4A. The distal end of the plastically deformable tabs 42 incorporate a taper 44 and a tab platform 46, such that when the tab platform 46 is engaged with the corresponding tab aperture 40 contained within the wall 30 of the outer member 14, distraction is locked and cannot be decreased. One advantage of this system include maximizing the available volume within the implant for graft thickness, as the distraction locking mechanisms comprising the plastically deformable tabs and the tab aperture allow for locked distraction mechanisms while minimizing wall thickness requirements for either the inner member 12 or the outer member 14.

During intended use, in embodiments of the invention, as the inner member 12 advances outwardly from the outer member 14, the tab platforms 46 of the one or more plastically deformable tabs 42 will move from one corresponding tab aperture 40 to the next as the expandable corpectomy implant 10 expands. Once a tab platform 46 abuttedly engages with a corresponding tab aperture 40 in a plane substantially orthogonal to the axis of expansion, with the face of the tab platform 46 facing a direction opposite to the direction of expansion abutting the portion of the frame of the tab aperture 40 facing the direction of expansion, the interaction during intended use will lock the expansion in place and prevent unintended collapse of the expandable corpectomy implant 10 in expanded form.

In at least one embodiment, the inner member 12 comprises two plastically deformable tabs positioned 180 degrees apart from each other (not shown). In varying embodiments, the inner member 12 may incorporate one or more plastically deformable tabs 42 positioned radially, and with their longest aspect of their bulk positioned substantially parallel to the height axis of the expandable corpectomy implant 10. Once distracted by an external instrument, in an embodiment, the expandable corpectomy implant 10 is locked into position axially by the interaction of the tab platform 46 of at least one plastically deformable tab 42 with the tab aperture 40 contained within the wall 30 of the outer member 14. For unlocking a subsequent reduction of distraction of the expandable corpectomy implant 10, an external instrument will be used to unlock axial distraction by placing inward pressure on the one or more plastically deformable tabs 42 contained within an expandable corpectomy implant 10, and thereby collapse the expandable corpectomy implant 10. In various embodiments, either or both the inner member 12 may incorporate apertures (optionally threaded) designed to accommodate set screws of a type and function recognized by one skilled in the art, which during intended use may tighten and lock distraction of the expandable corpectomy implant 10 and operate to prevent further sliding movement of the inner member 12 relative to the outer member 14.

In embodiments of the invention, the expandable corpectomy implant 10 comprises a locking system, comprising the plastically deformable tabs 42, each comprising the taper 44 and the tab platform 46. In embodiments, the taper 44 and tab platform 46 of the plastically deformable tabs 42 allow for the plastically deformable tabs 42 to freely move to interact with subsequent tab apertures 40 as the expandable corpectomy implant 10 expands axially, but not interact with previously encountered tab apertures 40 via movement in the opposite direction. The taper 44 of each plastically deformable tab 42 assumes a wedge shape that facilitates the deformation of each plastically deformable tab 42. In various embodiments, this action occurs during intended usage such that once the tab head reaches the edge of each aperture 40 located in the direction of expansion, the edge of the aperture 40 places force upon the taper 44 thereby bending the entire plastically deformable tab 42 inward within the inner member 12. Once the taper 44 of the plastically deformable tab 42 reaches the subsequent aperture fully, the taper 44 slides within the void of the tab aperture 40 and until the tab platform 46 located at the end of the plastically deformable tab 42 opposite the direction of expansion comes to rest against the frame of the tab aperture 40. One advantage that such a locking system embodies, which is that unlike locking systems of the prior art which require set screws and or rotating masses, the at least one embodiment of the present system's locking system requires much less mass, thereby minimizing the wall thickness requirements of the implant and thereby maximizing the available volume within the implant for the placement of biologic materials, such as bone graft.

One advantage of some embodiments of the present disclosure over prior art cages related to internal volume. Unlike prior art implants, which incorporate internal distracting and locking mechanisms thereby minimizing the available internal volume contained within, at least one embodiment is configured such that the distraction force is delivered primarily by mechanisms external to the expandable corpectomy implant 10. In some embodiments, this external distraction is made possible due to the locking mechanisms contained within the walls of the inner member and the outer member.

Embodiments of the present disclosure incorporate one or more lattice structure walls within the inner member 12 and outer member 14. In embodiments, the lattice structure forms the bulk of the walls 20 and 30, respectively, of both the inner 12 and outer members 14, respectively. One advantage associated with the use of additive manufacturing, as opposed to machining, to create the lattice structure. In at least one embodiment additive manufacturing is utilized to precisely place titanium to create the desired lattice structure construct. The lattice wall construct, forming the bulk of the walls 20 and 30, respectively, of the inner member 12 and the outer member 14, respectively, in embodiments of the invention, facilitates bone growth into and around the invention, and increases the volume available within the expandable corpectomy implant 10 for placement of biologic materials, such as bone graft.

One of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of present teachings. The descriptive labels associated with the numerical references in the figures are intended to merely illustrate embodiments of the invention, and are in no way intended to limit the invention to the scope of the descriptive labels.

The benefits, advantages, solutions to problems, and any element(s) that may cause any benefit, advantage, or solution to occur or become more pronounced are not to be construed as a critical, required, or essential features or elements of any or all the claims. The invention is defined solely by the appended claims including any amendments made during the pendency of this application and all equivalents of those claims as issued.

Moreover in this document, relational terms such as first and second, top and bottom, and the like may be used solely to distinguish one entity or action from another entity or action without necessarily requiring or implying any actual such relationship or order between such entities or actions. The terms “comprises,” “comprising,” “has”, “having,” “includes”, “including,” “contains”, “containing” or any other variation thereof, are intended to cover a non-exclusive inclusion, such that a process, method, article, or apparatus that comprises, has, includes, contains a list of elements does not include only those elements but may include other elements not expressly listed or inherent to such process, method, article, or apparatus. An element proceeded by “comprises . . . a”, “has . . . a”, “includes . . . a”, “contains . . . a” does not, without more constraints, preclude the existence of additional identical elements in the process, method, article, or apparatus that comprises, has, includes, contains the element. The terms “a” and “an” are defined as one or more unless explicitly stated otherwise herein. The terms “substantially”, “essentially”, “approximately”, “about” or any other version thereof, are defined as being close to as understood by one of ordinary skill in the art, and in one non-limiting embodiment the term is defined to be within 10%, in another embodiment within 5%, in another embodiment within 1% and in another embodiment within 0.5%. The terms “coupled” and “linked” as used herein is defined as connected, although not necessarily directly and not necessarily mechanically. A device or structure that is “configured” in a certain way is configured in at least that way, but may also be configured in ways that are not listed. Also, the sequence of steps in a flow diagram or elements in the claims, even when preceded by a letter does not imply or require that sequence.

While the present disclosure has been described herein in connection with certain embodiments so that aspects thereof may be more fully understood and appreciated, it is not intended that the present disclosure be limited to these particular embodiments. On the contrary, it is intended that all alternatives, modifications and equivalents are included within the scope of the present disclosure as defined herein. Thus, the examples described above, which include particular embodiments, will serve to illustrate the practice of the inventive concepts of the present disclosure, it being understood that the particulars shown are by way of example and for purposes of illustrative discussion of particular embodiments only and are presented in the cause of providing what is believed to be the most useful and readily understood description of procedures as well as of the principles and conceptual aspects of the present disclosure. Changes may be made in the formulation of the various compositions described herein, the methods described herein or in the steps or the sequence of steps of the methods described herein without departing from the spirit and scope of the present disclosure. Further, while various embodiments of the present disclosure have been described in claims herein below, it is not intended that the present disclosure be limited to these particular claims. Applicants reserve the right to amend, add to, or replace the claims indicated herein below in this or subsequent patent applications.

What is claimed:

1. An expanding spinal implant, comprising:
an inner member; and
an outer member;
said inner member comprising at least one plastically deforming tab,
said outer member comprising at least one tab aperture.

2. The expanding spinal implant of claim 1 wherein the inner member and the outer member have walls comprising a lattice structure.

3. The expanding spinal implant of claim 1 wherein the physically deforming tab comprises a taper and a tab platform.

4. The expanding spinal implant of claim 1 wherein the inner member and outer member have a selected cross sectional shape.

5. The expanding spinal implant of claim 1 wherein the at least one plastically deforming tab of the inner member engages the at least one tab aperature of the outer member such that the inner member and the outer member are locked to one another.

6. An expandable spinal implant, comprising:
an inner member having a first end, a second end and a sidewall; and
an outer member having a first end, a second end and a sidewall,
wherein the second end of the inner member is positioned in the first end of the outer member and wherein the inner member and the outer member have a selected cross sectional shape.

7. The expandable spinal implant of claim 6 wherein the inner member is configured such that it slidably protrudes from the outer member.

8. The expandable spinal implant of claim 6 wherein the inner member having at least one tab incorporated into the sidewall of the inner member and the outer member having at least one aperture formed in the sidewall of the outer member such that the at least one tab of the inner member engages the corresponding at least one aperture of the outer member so that the inner member is locked with the outer member.

9. The expandable spinal implant of claim 8 wherein the at least one tab comprises a taper and a tab platform.

10. The expandable spinal implant of claim 9 wherein the inner member and the outer member are movable to an expanded position wherein in the expanded position, the inner member advances outwardly from the outer member so that the tab platform of the at least one tab moves from the at least one corresponding tab aperture to the next at least one tab aperture.

11. The expandable spinal implant of claim 10 wherein once the tab platform abuttedly engages with a corresponding tab aperture in a plane substantially orthogonal to the axis of expansion between the inner member and the outer member, with the face of the tab platform facing a direction opposite to the direction of the expansion abutting the portion of the tab aperture facing the direction of expansion such that the interaction between the tab platform and the tab aperture locks the inner member and the outer member in place and prevents unintended collapse of the expandable corpectomy implant when in expanded form.

12. The expandable spinal implant of claim 10 wherein the inner member is provided with a fastener to prevent sliding movement of the inner member relative to the outer member.

13. The expandable corpectomy implant of claim 6 wherein the sidewall of the inner member and the sidewall of the outer member comprising a lattice structure.

14. The expandable corpectomy implant of claim 13 wherein a biologic material is implanted in the lattice structure.

15. A method of using an expandable corpectomy implant, comprising the steps of:
providing the expandable spinal implant, comprising:
an inner member having a first end, a second end and a sidewall; and
an outer member having a first end, a second end and a sidewall,
wherein the second end of the inner member is positioned in the first end of the outer member and wherein the inner member and the outer member have a selected cross sectional shape; and
inserting the expandable spinal implant in the selected portion of a spine of an individual.

16. The method of claim 15 further comprising the step of:
moving the inner member of the expandable spinal implant in a selected direction outwardly from the outer member of the expandable spinal implant so that at least one tab formed in the sidewall of the inner member of the expandable spinal implant engages at least one aperture formed in the sidewall of the outer member of the expandable spinal implant so that the inner member and outer member of the expandable spinal implant are in an expanded form.

17. The method of claim 16 further comprising the step of:
abuttedly engaging the tab of the inner member of the expandable spinal implant with the corresponding tab aperture of the outer member of the expandable spinal implant in a plane substantially orthogonal to the axis of expansion between the inner member and the outer member of the expandable spinal implant, with the face of the tab facing a direction opposite to the direction of the expansion abutting the portion of the tab aperture facing the direction of expansion such that the interaction between the tab and the tab aperture locks the inner member and the outer member of the expandable spinal implant in place and prevents unintended collapse of the expandable corpectomy implant.

18. The method of claim 16 further comprising the step of:
providing a fastener for the inner member of the expandable spinal implant; and
tightening the fastener to prevent a sliding movement of the inner member in relation to the outer member of the expandable spinal implant.

19. The method of claim 15 wherein the sidewall of the inner member and the sidewall of the outer member are comprised of a lattice structure.

20. The method of claim 19 further comprising the step of:
implanting a biologic material in the lattice structure.
